(19) United States
(12) Patent Application Publication
(10) Pub. No.: US 2023/0055910 A1
(43) Pub. Date: Feb. 23, 2023

(54) METHOD AND APPARATUS FOR DETERMINING DEPTH AND HEALTH OF PERIODONTAL SULCUS

(71) Applicant: Jeffrey M. Rosenberg, Ocean City, NJ (US)

(72) Inventor: Jeffrey M. Rosenberg, Ocean City, NJ (US)

(21) Appl. No.: 17/820,704

(22) Filed: Aug. 18, 2022

Related U.S. Application Data
(60) Provisional application No. 63/260,367, filed on Aug. 18, 2021.

(51) Int. Cl. A61B 5/00; A61C 9/00

(52) U.S. Cl. CPC A61B 5/4552; A61B 5/0095; A61C 9/0086; A61B 5/0035; A61B 5/682; A61B 5/0013; A61B 2576/02

(57) ABSTRACT

A non-invasive method for determining a gingival pocket depth includes seating a photoacoustic probe tray in a subject's mouth; transmitting photonic energy transgingivally; receiving generated ultrasonic signals; determining and processing the time of flight between transmitting and receiving and a relative amplitude of the ultrasonic signals to the transmitted photonic energy to determine densities and a topography of the subject's dental anatomy; determining a repeatable reference point; and measuring the gingival pocket depth in relation to the repeatable reference point. A system includes a transducer tray a biogel-containing bite wafer seated within the transducer tray; a processor; a memory; a user interface; and a visual display. The transducer tray has at least one embedded pulsed laser source and at least one embedded ultrasonic sensor.

### CROSS-REFERENCE TO RELATED APPLICATION

This application claims the benefit of priority of U.S. provisional application No. 63/260,367, filed Aug. 18, 2021, the contents of which are herein incorporated by reference.

### BACKGROUND OF THE INVENTION

The present invention relates to periodontal examination and, more particularly, to a method and apparatus for determining the depth and health of the periodontal sulcus.

Periodontal diseases (PD) are prevalent both in developed and developing countries, affecting about 20-50% of global population. There is a high prevalence of periodontal disease in adolescents, adults, and older individuals, making it a public health concern.

PD is likely to cause a 19% increase in the risk of cardiovascular disease, with an increase in relative risk of up to 44% among individuals aged 65 years and over. Type 2 diabetic individuals with a severe form of periodontal disease have 3.2 times greater mortality risk compared with individuals with no or mild periodontitis. Periodontal therapy has been shown to improve glycemic control in type 2 diabetic subjects. Periodontitis is related to maternal infection, preterm birth, low birth weight, and preeclampsia. Oral disease prevention strategies should be incorporated in chronic systemic disease preventive initiatives to curtail the burden of disease in populations. The reduction in the incidence and prevalence of periodontal disease can reduce its associated systemic diseases and can also minimize their financial impact on the health-care systems. It is hoped that medical, dental practitioners, and other health-care professionals will become familiar with the perio-systemic link and risk factors and the need to refer patients for dental and periodontal care. PD and gingival bleeding are highly prevalent in the adult population worldwide. World Health Organization (WHO) data shows that 90-100% of 34-year-old adults present gingival inflammation. Therefore, an investigation method is needed to allow assessment of the periodontal disease as well as monitoring of the evolution of gingival inflammation after periodontal treatments. Non-invasive and operator-independent methods for periodontal examination are necessary for diagnosing and monitoring the periodontal disease. Periodontal ultrasonography is a reliable technique for visualizing the anatomical elements which are necessary to diagnose periodontal status. Using this imaging technique, the dentino-enamel junction, the cortical bone, the radicular surface from the crown to the alveolar bone, and the gingival tissue can be seen without interfering with those elements during the examination. Also, calculus visualization is possible before and after scaling to evaluate the quality of the treatment. The prevalence of periodontitis has historically been measured using the extent and severity of loss of attachment and/or probing pocket depth in millimeters which represents an accretion of the manifestations of past disease with little or no indication of present disease activity. To best assess present disease activity, a dependable method of quantifying periodontal disease incidence is essential. Tools to measure periodontal diseases at the surveillance level have focused on current manifestations of disease (e.g., gingival inflammation) or disease sequelae (e.g., periodontal pocket depth or loss of attachment) but not progression of disease activity.

PD is associated with bacterial plaque buildup in the gingival sulcus (the gum line) and the body immune response to that bacterial challenge. Many indicators can signal ongoing periodontal destruction, including polymorphonuclear leukocyte (PMN) defects, high titers of antibodies against periodontopathogens, enzymes such as aspartate aminotransferase, measurement of subgingival temperature (e.g., a periodontal temperature probe such as the PerioTemp™ System) can indicate the existence of inflammation, but not necessarily provide information about the progression of the disease. None of the current methods for testing host response have yet achieved routine utilization in practice. Moreover, they are not considered a reimbursable expense by insurance companies.

The current metrics for monitoring periodontal health include attachment level, probing depth, bone loss, mobility, recession, and degree of inflammation. The gold standard for quantifying periodontitis progression is the measurement of clinical attachment loss with a manual periodontal probe (PP). This tool provides a numerical metric that reflects the degree of apical epithelial attachment measured from the gingival margin and is critical for disease staging. But the manual probe can only measure depth at the point of insertion—with no information on the full width or contour of the pocket. Furthermore, the benefit of traditional probing around dental implants is abrogated due to the implant threads that impede probe penetration along the implant surface. This limits clinical assessment of these tissues, potentially leading to periimplantitis. Recording of measurements is necessary to compare readings from one date to another see if the data is changing. Because this is a tedious and painful process that is seldom done accurately or to completion, it is difficult to accurately identify and track the progress of PD. The result is PD progresses insidiously and continually.

In addition to manual probing with the PP, another approach is disclosed in U.S. Pat. No. 5,755,571 to Companion. The probe disclosed in this patent uses intrasulcular ultrasound waves projected along the surface of the tooth for determining the periodontal pocket depth. This method is less invasive yet inaccurate and messy due to the instability of a handheld device that injects water into the periodontal pocket. In addition, the water stream theoretically can mobilize the bacterium and cause cross contamination of site. Furthermore, the probe disclosed in Companion requires a fixed reference point and uses the cementoenamel junction (CEJ) as that reference point. The CEJ is often difficult to locate or may be obliterated due to variation or restorative materials covering it.

A device known as the Florida Probe® uses a spring-loaded mechanism to detect the periodontal pocket. Conventional manual probing and the Florida Probe® are similar in that they invade the pocket to the base yet suffer from lack of repeatability and are often uncomfortable to the patient.

Rosenberg disclosed a method and apparatus in U.S. Publication No. 2006/0211954 A1 using ultrasound waves to scan, identify, and measure the gingival pocket using a tray-like design with biowafer coupler that retains the aqueous medium. This device is non-invasive and less disruptive than other devices. The coupler stabilizes the device for reproducibility. The device identifies the CEJ and references it for current and future scans/measurements. This device has a movable transducer that travels along the tray to generate the scan. This feature limited certain scan directions and complicated the stabilization of the device leading to various inaccuracies. The disclosure of U.S. Publication No. 2006/0211954 A1 is incorporated herein in its entirety.

Photoacoustic imaging is also known as a method for imaging the interiors of living organisms by utilizing the photoacoustic effect. Generally, in photoacoustic imaging, pulsed laser beams such as laser pulses are irradiated into the living organisms. Biological tissue that absorbs the energy of the pulsed laser beams generates ultrasonic waves (photoacoustic signals) by volume expansion thereof due to heat. The photoacoustic signals are detected by an ultrasound probe or the like, and the detected signals are analyzed, to enable visualization of the living organisms based on ultrasonic waves. The irradiation of the pulsed laser beam and the detection of the photoacoustic waves are repeatedly executed while shifting the probe element corresponding to single channels (single lines), to construct the photoacoustic image. The ultrasound probe may be equipped with elements capable of outputting ultrasonic waves (acoustic signals) into the biological tissue, detecting ultrasonic waves (“photoacoustic signals”) generated within the biological tissue by the pulsed laser beam being irradiated thereon, and detecting acoustic waves (“reflected acoustic signals”) which are the output ultrasonic waves reflected by the biological tissue. Each probe element converts the detected photoacoustic signals and the reflected acoustic signals into electric signals, and outputs the electric signals. See U.S. Pat. No. 8,650,960 to Tsujita.

US Publication No. 20210093200A1 to Jokerst disclosed use of a mouthpiece or mold made to fit over the periodontium with one or more photoacoustic and/or optical transducers and sensors that are disposed within or on the outer body of the mold to substantially align with the one or several teeth, or all teeth, in an arch when the outer body is inserted into a patient's mouth that generate imaging data from the one or more photoacoustic imaging sensors capable of being converted to an image of the periodontium or periodontal tissue, or an image of the periodontal pocket, or a periodontal probing depth image or measurement. This device lacks an inherent stabilizing factor making the ability to relate to a reference point variable and unpredictable. In addition, this device lacks a software component that “alerts” the user of historic changes and lacks the graphic depiction of the pocket needed to motivate patients to seek treatment.

As can be seen, there is a need for a non-invasive, predictable, and reproducible method of identifying and tracking PD and motivating patients to seek treatment.

### SUMMARY OF THE INVENTION

In one aspect of the present invention, a non-invasive method for determining a gingival pocket depth comprises seating a photoacoustic probe tray in a subject's mouth; transmitting photonic energy transgingivally; receiving ultrasonic signals generated thereby; determining a time of flight between the step of transmitting and the step of receiving; determining a relative amplitude of the ultrasonic signals relative to the amplitude of the transmitted photonic energy; processing the time of flight and the relative amplitude to determine densities of dental tissues in the subject's mouth and a topography of dental anatomy of the subject's mouth; determining a repeatable reference point; and measuring the gingival pocket depth in relation to the repeatable reference point.

In another aspect of the present invention, a non-invasive photoacoustic dental probe comprises a transducer tray with at least one pulsed laser source embedded therein and at least one ultrasonic sensor embedded therein; and a biogel-containing bite wafer seated within the transducer tray.

In yet another aspect of the present invention, a system comprises a transducer tray with at least one pulsed laser source embedded therein and at least one ultrasonic sensor embedded therein; and a biogel-containing bite wafer seated within the transducer tray; a processor electronically connected with the at least one pulsed laser source and the at least one ultrasonic sensor; a memory electronically connected with the processor; a user interface electronically connected to the processor; and a visual display electronically connected to the processor.

The present invention enables both numerical and graphical storage of the scan data. The present invention also enables storage of an individual's data for later comparisons and “alerts” the user of significant changes. Graphics software enables the patient to “see” what is occurring in their gum line for education and motivation.

The disclosures of the references discussed above are all incorporated herein by reference.

These and other features, aspects and advantages of the present invention will become better understood with reference to the following drawings, description, and claims.

### BRIEF DESCRIPTION OF THE DRAWINGS

FIG. 1 is a schematic view of a photoacoustic wave generator/ultrasound receiver device according to an embodiment of the present invention;

FIG. 2 is another schematic view of the transducer tray thereof;

FIG. 3 is a schematic cross-section thereof;

FIG. 4 is a schematic top view thereof;

FIG. 5 is a schematic view of a scan area in a method according to an embodiment of the present invention;

FIG. 6 is a schematic view of dental anatomy;

FIG. 7 is a schematic cross-sectional view thereof;

FIG. 8 is a schematic view of a system according to an embodiment of the present invention; and

FIG. 9 is a flow chart of a scanning method according to an embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a non-invasive means for determining the depth of a gingival pocket using photoacoustic and ultrasound signals transmitted transgingivally and through the gingival sulcus. Embodiments of the present invention include a method of photoacoustic scanning, a scanning apparatus therefor, and a system comprising the scanning apparatus.

Ultrasound operates under the principle of “sound in, sound out”, but photoacoustic imaging shifts this concept to “light in, sound out”. Ultrasound waves easily travel through tissue, providing a much more in-depth view, but do not have the ability to discern a tissue's chemical components and therefore do not capture important information conveyed by light-based imaging. Photoacoustic imaging combines the abilities of multiple imaging techniques into one platform. It uses extremely short laser or light bursts that safely cause cells or other light absorbers to emit ultrasound waves, which then travel unimpeded back through the tissue to sensors that use a detection system to translate the signal into an image.

Transgingival (through the gums) transmission of the photoacoustic and ultrasonic signals (the scan) permits a completely non-invasive determination of the depth of the gingival sulcus (gumline) along with a point of reference used to determine if there is loss of the gingival attachment apparatus. In addition, the inflammatory “status” of the sulcus may be determined with photoacoustic imaging. Disclosed herein are various embodiments for ultrasonic and photoacoustic imaging.

The present invention includes a method, which in its broadest sense, comprises transmitting a photoacoustic signal through the gum tissue of the individual (transgivally) from the buccal/facial direction, the palatal/lingual direction, and intra-sulcularly from an occlusal/incisal direction with a photoacoustic transducer probe and receiving signals emitted by and reflected from the gum tissues and underlying structures with photoacoustic sensors. The time of flight of the reflected signals and their relative amplitude may be used to determine the topography of the underlying structures. The method may achieve results comparable to dental cone beam computed tomography but without ionizing radiation.

The inventive method comprises identifying a repeatable reference point, measuring the gingival pocket depth with relation to the reference point, and assessing the presence or absence of inflammation in the gingival pocket. The photoacoustic and ultrasound waves are both emitted in plurality through a mouth-shaped tray transducer with embedded silicon chip material that is coupled to the oral tissues with a chemically coated envelope housing a colloidal biogel (a biowafer). The tray may be configured to fit in the mouth and scan a full row of teeth (i.e., all of the upper teeth or all of the lower teeth) or the tray may be sectional in nature, configured to scan teeth one to several teeth at a time. The transducer may be configured to emit waves in a phased array. The signals are then reflected off the shapes of the gingival pocket and returned in a multitude of raw wave forms. Coating the bladder with a photoactive material such as disclosing dye (which has been used in dentistry for decades) enables photoacoustic images to be imaged in greater detail, much crisper and better defined than conventional ultrasound photoacoustic computer tomography (PACT) alone, when irradiated with a laser. These wave forms are processed, with the aid of artificial intelligence, into processed waveforms. The time of flight between the transmission of the photoacoustic waves and reception of the reflected waves is measured. The amplitude of the reflected signals is measured and the density, and shape of the material from the reflected signals is determined. As such, the densities of the various dental tissues may be determined. Software interpretation of the processed waves (e.g., with artificial intelligence operative to process waveforms, for example using machine learning) may be utilized to convert the data to depth measurements. A visual display presents graphical images to show these measurements and shapes for education and motivation. The disclosing dye of the biowafer discloses dental plaque which is also informational and motivational. The frequency range is not particularly limited. For example, the frequency may be up to around forty megahertz (MHz).

One or more wireless transmitters and/or receivers are used for transmitting data signals generated by the transducers and/or for receiving control signals to operate the light generators. In an example, a tray transducer may have a wireless [e.g., a radio frequency (RF)] transmitter of any suitable type, transmitting some or all of the generated data signals to a detection system, such as may be embodied in a processing device.

The processing device may comprise one or more processors, a memory or memories, wired and/or wireless interface(s), and suitable computer-readable instructions stored on a non-transitory medium or media operative to cause the processor to receive and process image data signals and to generate an image as disclosed herein. The system may further comprise one or more databases. In some embodiments, the processing device may be configured to receive data signals (e.g., locally), to store the received data signals, and/or to forward (wired or wirelessly, via a network, the Internet, etc.) the received data signals to an operatively linked remote receiver, such as another processing device. The processing device may generate both a photoacoustic image and an ultrasound image of the periodontium, or portion thereof; and overlay the images to produce a composite image.

The system comprises one or more photoacoustic imaging sensors and corresponding transducers. The photoacoustic imaging sensors comprise ultrasound and/or optical imaging sensors, capable of generating imaging data, that are linked to the corresponding ultrasound and/or optical transducers. The transducers are operative to transmit the imaging data to a remote receiver which generates images of the periodontium. The sensors generate imaging data of the facial/buccal (front), lingual/palatal (back) and occlusal regions, moving in sequential manner around the dental arch. The transducers may generate additional ultrasound imaging data of the periodontium or a portion thereof.

According to some embodiments of the present invention, an apparatus for executing the method is provided. In its broadest sense, the apparatus comprises a photoacoustic probe comprising a transducer for emitting light energy and ultrasound signals and a photoacoustic and ultrasound sensor for acquiring a reflected signal. The transducer may emit ultrasound with a frequency within the range of about 20 MHz to about 40 MHz. The apparatus further comprises a pulsed transmitter or pulsed laser source for generating photoacoustic signals, a multi-surfaced ultrasonic detector, and a fast data acquisition system that can triangulate the origin of an ultrasonic wave from the underlying dental structures. For example, photoacoustic imaging may be performed by pulsing light through two optical fiber bundles integrated with the transducer tray. The laser excitation (Q-switched Nd:YAG) may use, e.g., 5 ns pulses at 20 Hz (6 Hz frame rate). Alternatively, light-emitting diodes (LEDs) may be disposed within or on a gum-facing surface of the tray. The laser sources are disposed and configured such that light signals, such as light pulses, emanate from their source and penetrate any contrasting agent, ultrasound medium, and/or bladder containing the ultrasound medium when the tray is fitted over the one or more teeth. They may be driven by a suitable driving circuit (not shown) coupled thereto. The light pulses may have a frequency of about 5 to 5,000 pulses per second.

The detection system is known in the art. Two instruments that may quantify acoustic signal are the NEXUS128™ (Endra Life Sciences, Ann Arbor, Mich.), and the VEVO™ 2100 (Fujifilm VisualSonics, Inc., Toronto, Canada).

The acoustic signal may be produced by one or more photoacoustic probes. Capacitive micro-machined ultrasonic transducer (CMUT) arrays may be used to detect the acoustic signal. In general, the use of photoacoustic signals to elucidate the internal structures of the human and the live subjects takes advantage of the fact that the degree to which a photoacoustic wave is absorbed, propagated, or reflected by any structure is a function of the density of that structure.

The inventive oral tray transducer with photoacoustic transducers and corresponding photoacoustic sensors embedded therein generates a phased array of photoacoustic and ultrasound signals that are coupled to areas to be scanned with a disposable gelatinous bladder. The entire dental arch may be scanned, recorded, and referenced for comparative analyses. In addition, a graphic of the scan may be used for patient education and motivation to address their periodontal health or disease.

The disposable gelatinous bladder comprises a disposable gelatinous coupling material enclosed in a chemically coated envelope/bladder form, a “biowafer”, and is used in a tray-shaped transducer that fits over the dental arch and periodontium for stabilizing and signal transmission of the device during the scan. An encapsulated bladder with a hydrogel such as Diacrylate Pluronic F127 (F127DA) contains micelles for use as macro-cross-linkers and drug carriers creating a “biogel.” The developed micelle-cross-linked biogel has superior mechanical properties to stabilize the tray and may be formed to fit the dental arch and the teeth to be measured. The biogel may be an acoustic coupling medium.

In another embodiment, the present invention provides a wave generator using photoacoustic technology to convert light absorption at the target depth to sound waves which scatter for transmission back to the surface. A nanosecond laser, at a predetermined wavelength, is directed at the gumline. The thermoelastic expansion of the tissue converts photons to sound waves which are used to form images with a resolution associated with the ultrasound frequency along the gingival pocket. This photoacoustic wave generator uses light to create and transmit photonic energy for higher spatial resolution. The photoacoustic wave generator may be modified to reveal the use of oxygen by the tissues and to detect inflammatory activity during a scan. Changes in depth and inflammation compared to previous scans are sent as an “alert” to scan data.

In some embodiments, the present invention provides a device used for both clinical and patient education purposes that seeks to improve motivation and access to treatment of periodontal disease.

The inventive method is believed to be more accurate than the current manual probes and easier for clinicians to use, increasing clinician compliance to identify and measure PD. This painless method may reduce patient fears regarding examination of the gums and make the public more likely to seek out appropriate care and obtain early diagnosis and treatment, thereby increasing public health and reducing morbidity given the overwhelming evidence of the relationship of PD to systemic diseases.

It is contemplated that embodiments of the present invention may have multiple applications in dentistry and medicine. The baseline establishment of the periodontal attachment and the progressive changes that occur over time provide insight to a diagnosis of healthy or diseased periodontal tissues. Detection of inflammation in the periodontal tissues may assist in determining whether the patient is in an active or inactive state of disease. Since periodontal inflammation is a co-factor in many systemic illnesses, especially cardiovascular disease and diabetes, embodiments according to the present invention may be used in general medicine, cardiology, and endocrinology to assist in patient screenings as a co-factor in systemic illnesses. Graphical depiction of the periodontal tissues may educate and motivate patients to seek care when needed.

The inventive system revolutionizes PD diagnosis and treatment by telling the clinician(s) and patient when and where to treat PD very early and very accurately. Further, because of public familiarity with other use of ultrasound, embodiments according to the present invention may lead to an increased demand for services and insurance reimbursements. The inventive method and system may also increase public awareness of PD and the need for treatment.

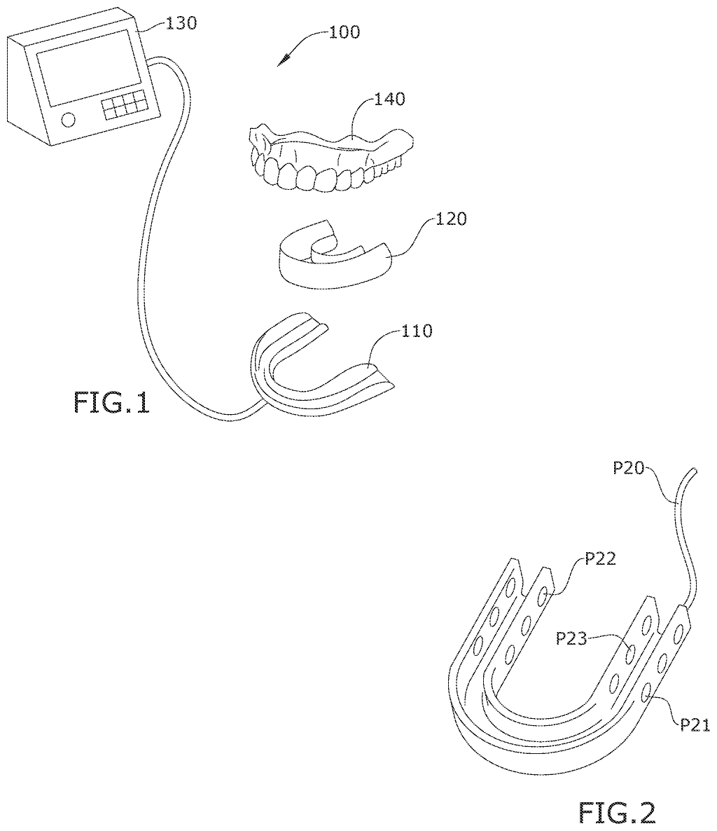

Referring to FIGS. 1 through 9, FIG. 1 illustrates a dental apparatus 100, comprising a transducer tray 110, with a bite wafer/coated bladder 120 inserted therein, electronically connected to a processing unit 130. The dental apparatus is used to determine the depth and health of the periodontal sulcus of a subject's dental anatomy 140.

As shown in FIG. 2, the tray 110 has embedded transducer silicon chips P21, P22, P23 and may be connected to the processing unit 130 via a wire P20. The processing unit 130 may comprise a non-transitory medium storing computer readable instructions and/or an imaging system controller including a pulser receiver, a processor card operative to run at least one algorithm, a graphic convertor, and an alert system.

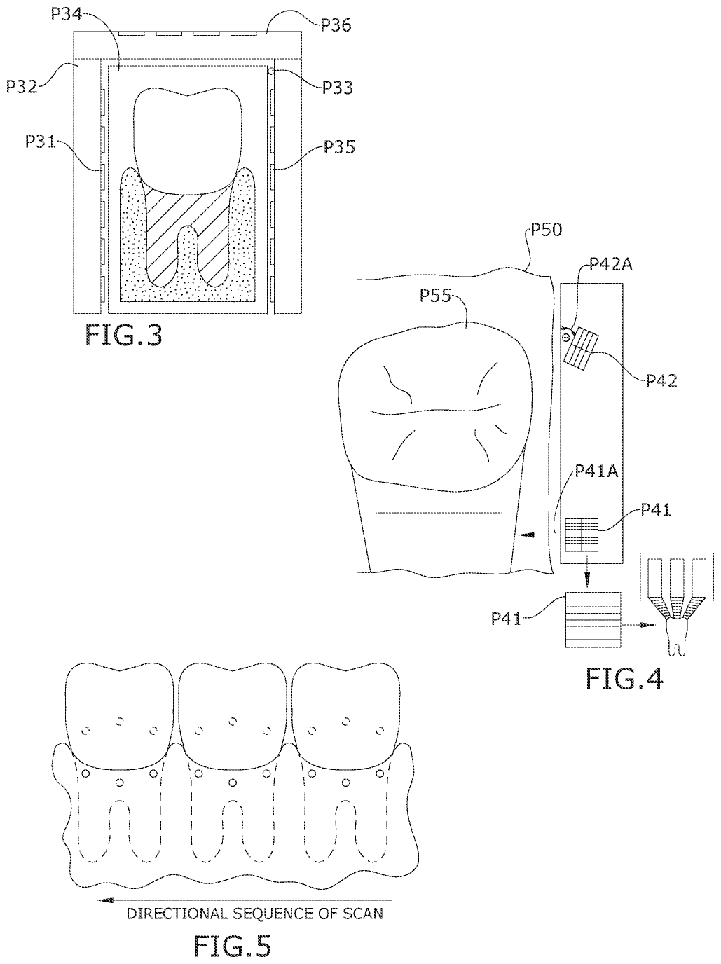

A cross-sectional view of the tray 110 is shown in FIG. 3. The tray comprises a transducer exoskeleton P32 with silicon chips P31, P33, P35, P36 embedded on a first surface, a second surface, a third surface, and a fourth surface. Ultrasonic detectors are thus present on multiple surfaces. The transducer may also have photoacoustic energy sources such as fiber optics or light emitting diodes. The transducer exoskeleton P32 surrounds a disclosing dye layer P34.

FIG. 4 illustrates first embedded laser or ultrasound energy source P41 emitting a wave in a first direction P41A through a coated biowafer bladder P50, and a second embedded laser or ultrasound energy source P42 positioned at an angle P42A, to determine the periodontal sulcus depth along a tooth P55 surface. FIG. 4 also schematically shows that more than one energy source P41 may be arrayed around the tooth to measure multiple points simultaneously.

The tray 110 transducer scans the teeth in a directional sequence, as shown in FIG. 5.

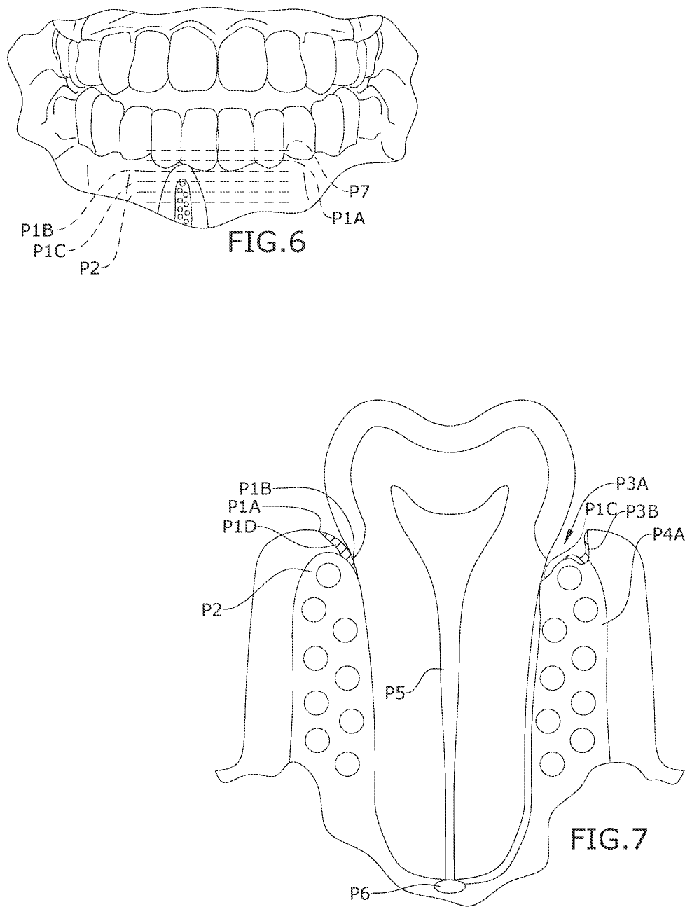

A front view of a subject's mouth is shown in FIG. 6, illustrating the relevant anatomy, including a gum tissue line P1A, a cementoenamel junction (CEJ) point P1B (which serves as a repeatable reference point), a base of the gingival sulcus P1C, an osseous bone structure P2, and tooth enamel P7. FIG. 7 provides a cross-section of a tooth within the mouth, illustrating the gingival sulcus P3A, a tissue lining P1D, a jawbone P4A with an infrabony crater P3B, tooth pulp tissue P5, and an apical area P6.

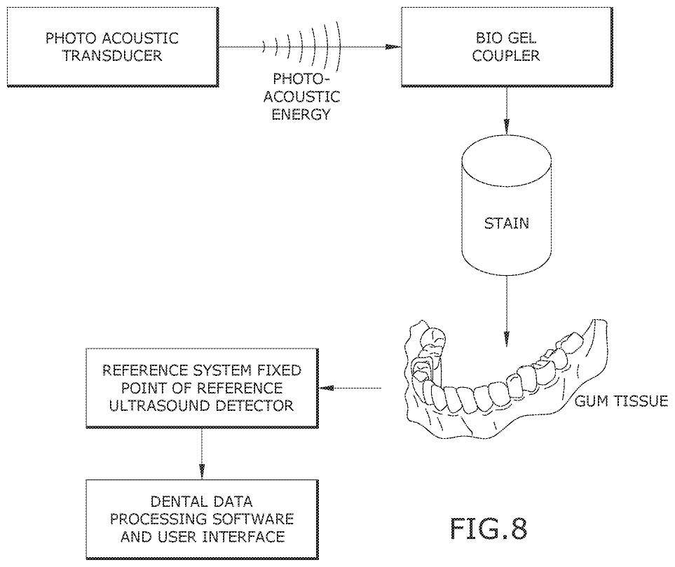

FIG. 8 illustrates a system according to an embodiment of the present invention. A tray has a photo acoustic transducer embedded therein and includes a bio gel (polymer coating) coupler wafer with disclosing dye or stain. An ultrasound detector/receiver also embedded into the tray has a reference system that identifies a fixed point of reference with respect to a subject's gum tissue. The photo acoustic transducer and ultrasound receiver may be operated with commercially available dental data processing software via a user interface.

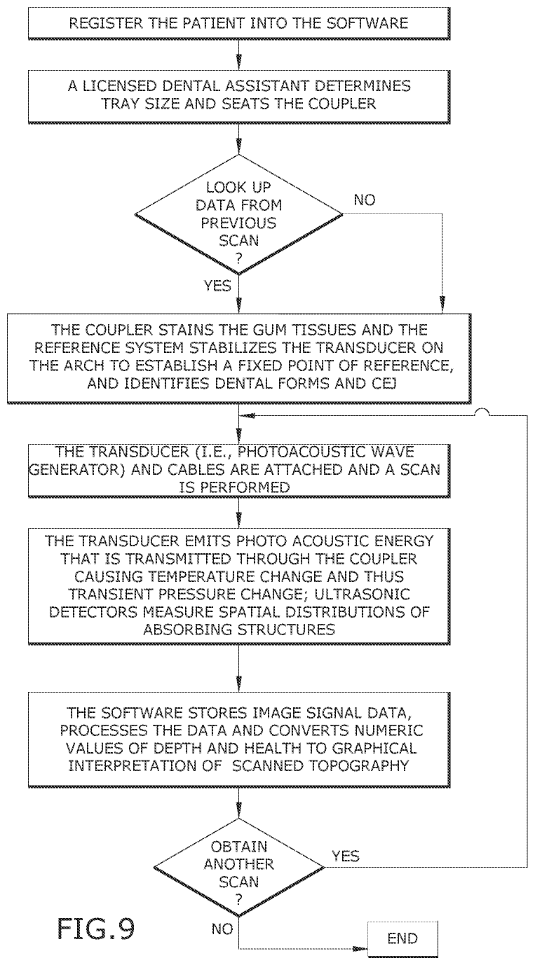

FIG. 9 illustrates a method of scanning a subject's teeth according to an embodiment of the present invention. A dental assistant determines a tray size suitable to the subject's mouth with the gel pad in place (small, medium, large; may be different for the upper teeth than the lower teeth) and seats the tray. The tray may be interfaced with dental software via a TWAIN interface. A reference system identifies dental forms and the tooth CEJ. The assistant may look up data from a previous scan for reference. The transducer generates a pulsed photoacoustic wave signal. When the transducer emits energy (e.g., laser light pulses), the beam is protected across the tooth and gumline and beyond. The subject's dental anatomy absorbs photons, leading to thermalization and a localized temperature increase which causes thermal expansion at the site of absorption. The thermal expansion creates a transient pressure change that is detected by an ultrasound detector that measures spatial distributions of the absorbing structures. The detected image signal data is filtered and stored, and the scan results are converted to numeric data and a graphical interpretation, providing an image reconstruction with reconstructed measurement values. The reconstruction data is the stored on a data storage device. In some cases, the assistant may perform another scan.

It should be understood, of course, that the foregoing relates to exemplary embodiments of the invention and that modifications may be made without departing from the spirit and scope of the invention as set forth in the following claims.

What is claimed is:

1. A non-invasive method for determining a gingival pocket depth, comprising:
seating a photoacoustic probe tray in a subject's mouth;
transmitting photonic energy transgingivally;
receiving ultrasonic signals generated thereby;
determining a time of flight between the step of transmitting and the step of receiving;
determining a relative amplitude of the ultrasonic signals relative to the amplitude of the transmitted photonic energy;
processing the time of flight and the relative amplitude to determine densities of dental tissues in the subject's mouth and a topography of dental anatomy of the subject's mouth;
determining a repeatable reference point; and
measuring the gingival pocket depth in relation to the repeatable reference point.

2. The non-invasive method of claim 1, further comprising:
transmitting ultrasonic signals transgingivally;
receiving reflected ultrasonic signals;
determining a second time of flight between the step of transmitting ultrasonic signals transgingivally and the step of receiving reflected ultrasonic signals;
determining a second relative amplitude of the reflected ultrasonic signals relative to the amplitude of the transmitted ultrasonic signals; and
processing the second time of flight and the second relative amplitude to determine densities of dental tissues in the subject's mouth and a topography of dental anatomy of the subject's mouth.

3. The non-invasive method of claim 1, further comprising assessing the dental anatomy to determine a presence or absence of inflammation in the gingival pocket.

4. The non-invasive method of claim 1, wherein the photonic energy is transmitted from a direction selected from the group consisting of: a buccal/facial direction, a palatal/lingual direction, intra-sulcularly from an occlusal/incisal direction, and any combination thereof.

5. The non-invasive method of claim 1, further comprising converting the time of flight and relative amplitude to a graphical image.

6. The non-invasive method of claim 2, further comprising converting the time of flight and relative amplitude to a first graphical image, converting the second time of flight and the second relative amplitude to a second graphical image, and overlapping the first graphical image and the second graphical image.

7. A non-invasive photoacoustic dental probe, comprising:
a transducer tray with at least one pulsed laser source embedded therein and at least one ultrasonic sensor embedded therein; and
a biogel-containing bite wafer seated within the transducer tray.

8. The non-invasive photoacoustic dental probe of claim 7, wherein the transducer tray further comprises at least one ultrasonic signal source embedded therein.

9. The non-invasive photoacoustic dental probe of claim 7, wherein the biogel-containing bite wafer is coated with a photoactive material.

10. The non-invasive photoacoustic dental probe of claim 7, wherein the at least one ultrasonic sensor is present on multiple surfaces of the transducer tray.

11. A system comprising:
a transducer tray with at least one pulsed laser source embedded therein and at least one ultrasonic sensor embedded therein; and
a biogel-containing bite wafer seated within the transducer tray;
a processor electronically connected with the at least one pulsed laser source and the at least one ultrasonic sensor;
a memory electronically connected with the processor;
a user interface electronically connected to the processor; and
a visual display electronically connected to the processor.

12. The system of claim 10, wherein the transducer tray further comprises at least one ultrasonic signal source embedded therein, electronically connected with the processor.

13. The system of claim 10, wherein the processor comprises artificial intelligence operative to process waveforms, thereby triangulating an origin of an ultrasonic wave from underlying dental structures, and software operative to convert data to depth measurements and graphical images.

14. The system of claim 12, wherein the processor comprises artificial intelligence operative to process waveforms, thereby triangulating an origin of an ultrasonic wave from underlying dental structures, and software operative to convert data to depth measurements and graphical images; and wherein the software is further operative to overlay the graphical images.

15. The system of claim 10, wherein the transducer tray further comprises a wireless transmitter and/or receiver.

16. The system of claim 10, wherein the processor further comprises a wireless transmitter and/or receiver.
